(19) United States
(12) Patent Application Publication
(10) Pub. No.: US 2024/0122783 A1
(43) Pub. Date: Apr. 18, 2024

(54) ROBOTIC THERAPY UNIT WITH ARTIFICIAL INTELLIGENCE INTEGRATED FEATURES FOR ACCOMPLISHING MUSCLE LENGTHENING

(71) Applicant: Benjamin J. Blankenship, Troy, MI (US)

(72) Inventor: Benjamin J. Blankenship, Troy, MI (US)

(21) Appl. No.: 18/384,505

(22) Filed: Oct. 27, 2023

Related U.S. Application Data
(63) Continuation-in-part of application No. 17/592,578, filed on Feb. 4, 2022.
Application No. 17/592,578 is a continuation-in-part of application No. 16/380,112, filed on Apr. 10, 2019, now abandoned.
(60) Provisional application No. 62/655,551, filed on Apr. 10, 2018.

(51) Int. Cl. A61H 7/00; G16H 20/30; G16H 40/67; G16H 80/00

(52) U.S. Cl. CPC A61H 7/00; G16H 20/30; G16H 40/67; G16H 80/00; A61H 2201/5015; A61H 2201/5046

(57) ABSTRACT

A robotic system, assembly and computer assisted media for providing therapeutic treatment not limited to muscle lengthening. An adjustable probe is mounted to a carriage, in turn supported in width and depth extending relationship upon a portable trolley supported frame or other movable carriage which can be positioned over a patient support device for applying treatment to a given patient muscle areas according input parameters selected from any of heat, cold, vibration (frequency), pulse pressure and duration. A body scanner is provided which interfaces with a processor input incorporating AI assisted software for any of a PC, tablet, laptop or smartphone with mobile application communicating with the software component for providing directions to actuate the probe to apply a treatment protocol. The input can be communicated remotely via NFC, Bluetooth or Cloud capabilities with a remote care provider or ACO organization.

### CROSS REFERENCE TO RELATED APPLICATIONS

The present application is a continuation in part of U.S. Ser. No. 17/592,578 filed Feb. 4, 2022. The '578 application is a continuation in part of U.S. Ser. No. 16/380,112 filed Apr. 10, 2019. The '112 application claims the priority of U.S. Ser. No. 62/655,551 filed Apr. 10, 2018.

### FIELD OF THE INVENTION

The present invention is directed to a system, method and software based system for accomplishing therapeutic lengthening of muscles associated with a robotic therapy unit.

### BACKGROUND OF THE INVENTION

The prior art is documented with examples of muscular therapy devices. A notable example of this is the robotic muscular therapy system of Meilus, U.S. Pat. No. 6,267,737 and which teaches applying repeated amounts of concentrated pressure to targeted muscles selectively to lengthen muscle tissue layer by layer and thereby reduce limitations on joint extension and flexibility as well as to eliminate pain caused by excess muscle contraction. Features associated with this device include a beveled treatment probe designed to concentrate pressure without breaking the skin of an average patient, a probe column assembly for fine X, Y, and Z probe movement over a patient, and a plurality of interchangeable column assembly supports for coarse X, Y, and Z probe movement. Patient safety limitations include a torque-limited and current-limited motor with a slip clutch, which causes the probe to retract from its treatment position when a patient actuates or when a pre-set maximum tissue pressure is encountered.

Other features include a swivel fitting which allows the probe to give/deflect, such as upon a patient sneezing or making another sudden movement, and thus allows patients to easily push the probe away upon demand. The system may optionally have each of an X-Y position-able patient support; automated control means probe movement; a computer learning mode for creating individualized treatment routines; patient movement sensors; and probe sensors for patient progress data collection. Applications can include elimination of acute and chronic of pain; treatment of conditions resulting from accidents and injury; pre-surgery conditions involving muscle spasm; post-surgery recovery, reduction of scar tissue, and restoration of flexibility; reduction of stress and tension; improved sports performance; treatment of conditions involving restricted physical movement; and postural improvement.

### SUMMARY OF THE PRESENT INVENTION

The present invention discloses an improved muscle lengthening method, assembly and computer writeable media for use with a robotic system and for providing customized treatment of a given patient. The present invention incorporates a novel probe and multi-axial adjustable carriage design, this in combination with a combination software enabled processor and display for enabling any from of customized treatment protocol to be communicated to the probe.

The system includes an adjustable probe head mounted for three dimensional adjustment along a carriage, such including multi-axial manual or (optionally) numerically controlled pre-position adjustability of the probe head. The probe can be supported in an underside extending relationship upon a portable trolley supported frame or other fixed or movable carriage which can be positioned over a patient support device (such as a bed, treatment table or the like) for applying treatment intervals to given patient muscle areas according input parameters selected from heat, cold, vibration (frequency), pulse (including percussion and tapotement), pressure and duration.

The probe head is operated by the software system integrated into such as a single board processor or other processor input, such not limited to any of a PC with display mounted to the carriage supporting the adjustable probe. Any of educational, marketing or entertainment features can also be integrated into the attached screen.

The present system also provides the ability to complete SOAP notes (an acronym for subjective, objective, assessment, and plan) and which is a method of documentation employed by health care providers to write out notes in a patient's chart, along with other common formats, such as an admission note. In this manner, the associated process component (including without limitation such as a touch screen display) provides the user with the ability to extract data to populate medical notes or other data to any external software systems, network devices or the like.

Other variants can include the use of any form of tablet, laptop or phone, such potentially being separately mounted to the support carriage and/or provided with a mobile application in communication with the software component, for providing any of NFC, Bluetooth or Cloud based directions to an associated numeric (NC) controller incorporated into the robotic system in order to program the probe to apply a treatment protocol according to the given combination of the heat, vibration, pressure, pulse and time interval protocols. In this fashion, the probe head works in combination with a best practices protocol programmed into the associated software component, such as which is tailored to provide a desired treatment to a patient according to determined medical standards.

Other aspects of the present design include the ability of the affixed or separate remote communicating processor to store and/or share secure patient records which may include but are not limited to treatment notes, and the like. Data sharing can further envision the use of any one or more of Cloud based, Messenger, USD/SD card, Mapping or 3D scanning options. The input/output aspects of the processor component associated with the probe can be communicated remotely via any of NFC (Near Field Communication), Bluetooth or Cloud capabilities with a remote care provider or ACO (or Accountable Care Organization which are defined as one or more groups of doctors, hospitals, or other health care providers, to provide coordinated care to a given patient group.

The practical outcomes of such treatments made possible by the present assembly include, without limitation, such as the dilation of blood vessels (vasodilation), such as in order to decrease blood pressure, realigning bone structure, breaking up adhesions, scar tissue and the like, and interrupting the physiological nervous response to defensively tighten/shorten the muscles.

Additional features include the incorporation of a body scanner into the carriage assembly, this working in combination with AI (artificial intelligence) features incorporated into the processor for conducting an initial body scan of the patient, at which point the processor assembles and outputs each of a detailed scan result and associated treatment protocol. The body scanner can be located along any of the front or side of the carriage for taking a standing scan of the patent, as well as relocated in an underneath and downward extending direction for scanning a patient laying atop a support gurney.

The AI features further can be incorporated into the mobile application and software components previously described for both operating the probe head, this including introducing real time updated protocols during the therapy sessions, as well as for permitting remote access of the data, with software for assisting in auto-population of notations and records. This functionality also allows for generating the SOAP notes as well as interfacing with electronic media records (EMR's) or other medical software.

### BRIEF DESCRIPTION OF THE DRAWINGS

Reference is now made to the attached drawings, when read in combination with the following detailed description, wherein like reference numerals refer to like parts throughout the several views, and in which:

FIG. 1 is a title screen illustration of an associated software component incorporated into the robotic therapy device and which illustrates a user login screen with ID and password fields;

FIG. 2 is a treatment set up screen succeeding the login screen and which includes a plurality of touch entry screens for establishing parameters for pressure, treatment duration, vibration percentage, and heat, along with start, stop, manual adjustment and logout fields;

FIG. 3 is a perspective view of a transportable trolley with width adjustable carriage upon to which is supported the robotic unit with adjustable probe head and numerically controlled actuating components according to one non-limiting embodiment of the present invention;

FIG. 4 is a front plan view of the trolley displaceable carriage and robotic unit of FIG. 3;

FIG. 5 is a rear plan view of the trolley displaceable carriage and robotic unit and further depicting the patient emergency stop button for deactivating operation of the therapy probe;

FIG. 6 is a cutaway taken along line 4-4 of FIG. 4 and showing the tightening knob for adjusting the lateral positioning of the carriage supporting the robotic unit with adjustable probe;

FIG. 7 is an illustration of the patient therapeutic probe head attached to the extending end of the robotic unit;

FIG. 8 is a front plan view illustration of a trolley displaceable carriage and robotic unit according to a further variant presenting a pair of carriage supported robotic units with adjustable probes along with a height adjustable trolley frame;

FIG. 9 is a cutaway taken along line 9-9 of FIG. 8 and illustrates the eccentric adjustability of the probe relative to an underside extending base support of the carriage adjustable robotic unit;

FIG. 10 is an illustration of an alternate adjustment mechanism in comparison to that shown in FIG. 9 and depicting first and second rotational or swivel connections associated with the carriage adjustable robotic unit and for adjusting a positioning of the probe relative to the patient being treated;

FIG. 11 is an illustration similar to FIG. 3 of a portable therapy unit according to a further non-limiting and preferred embodiment transportable trolley with width adjustable robotic unit with adjustable probe head and numerically controlled actuating components according to a further non-limiting embodiment of the present invention;

FIG. 12 is a front plan view of the unit of FIG. 11 and better showing the configuration of the multi-axial adjustable robotic unit of FIG. 3;

FIG. 13 is an enlarged and upper rear side perspective of the processor component mounted atop the width extending support along which the downwardly suspended robotic unit is adjustable

FIG. 14 is a front plan view similar to FIG. 12 and depicting a pair of carriage supported robotic units with adjustable probes along with a height adjustable trolley frame;

FIGS. 15-18 substantially repeats FIGS. 11-14, respectively, while depicting some updated features of the portable therapy unit including the shaping and construction of the multi-articulating arm;

FIG. 19 is an enlarged plan cutaway of the robotic therapy unit providing multiple axes of adjustment and further illustrating the heater wires extending from the probe head to the processor and display;

FIG. 20 is an illustration of a first version of a body scanner attached to a first location of the robotic therapy unit and which interfaces with AI functionality in order to assemble a treatment protocol;

FIG. 21 is a further illustration of a second version of a body scanner attached to a downward location of the therapy unit scanner for conducting a scan of a patient lying on a gurney located underneath the associated carriage; and

FIG. 22 presents a third variant in which the scanner is positioned at a side location of the robotic therapy unit carriage.

### DETAILED DESCRIPTION OF THE PREFERRED EMBODIMENTS

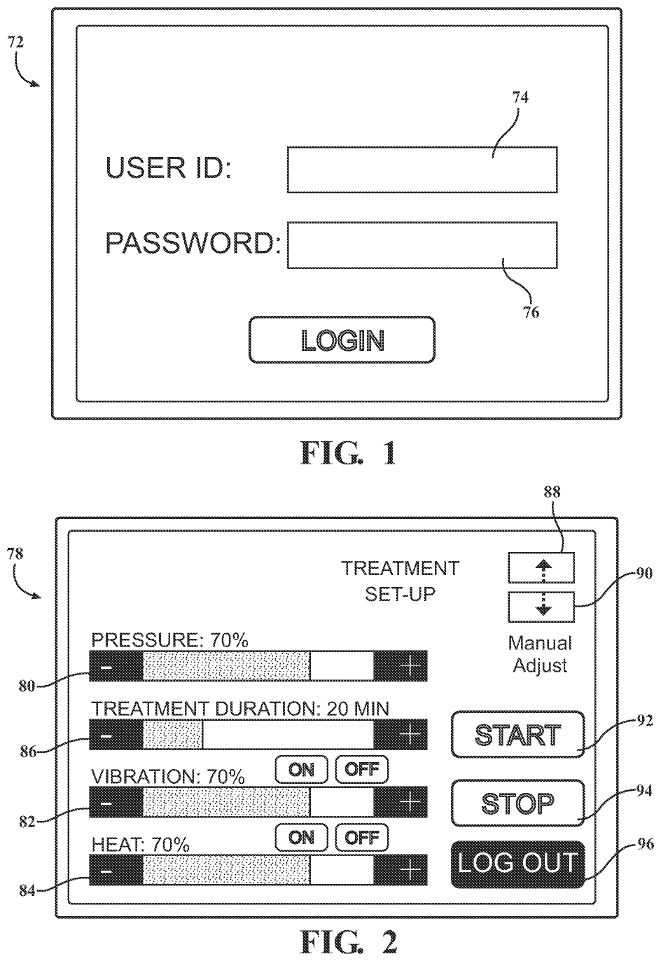

With reference to FIGS. 1-10, the present invention discloses a robotic therapy unit and associated method and processor/software component for providing tailored treatment protocols for lengthening patient muscles, As will be further described in more detail, the present invention is an improvement over prior art teachings for muscle lengthening including the ability to provide for customized treatment protocols, such as which can provide any or all of cold, heat, vibration, percussion/tapotement inputs for given time durations.

Also described below in further detail is the associated processor control and screen display aspects of the robotic therapy unit which provide the ability to customize the treatment provided, either by the technician/care provider tending to the patient, as well as can be further assisted by any remote located care provider/medical professional who has the ability to input patient treatment protocols via any of Bluetooth, Wireless NFC or Cloud protocols. As is further known, muscular tissue can shorten a variety of ways. A few examples would include repetitive motion in an occupation or sport, injury, spasm, autoimmune disease and many others. In one non-limiting application according to the below description, the robotic system, method and computer assisted module reverses this shortening and restores balance to the bone structure as much as the patient's condition will allow.

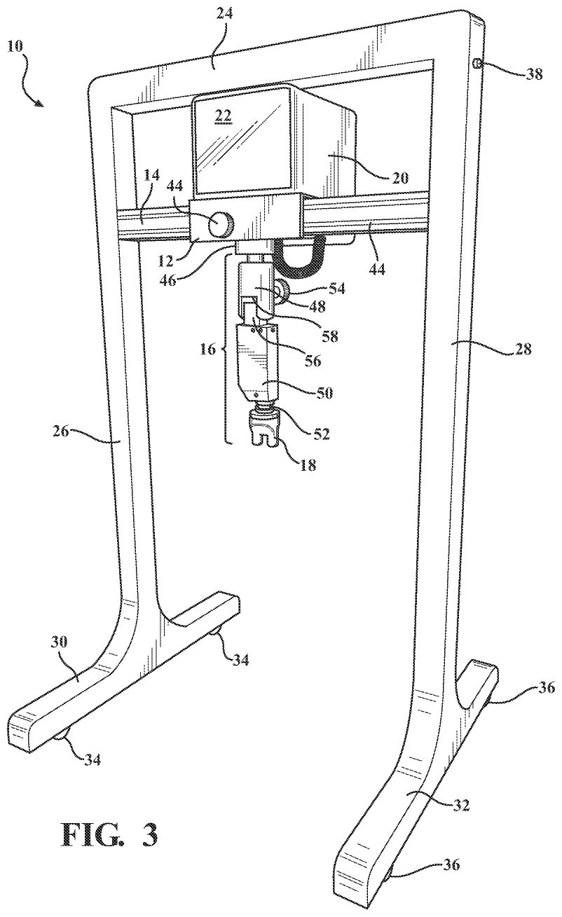

Referring initially to FIG. 3, a perspective view is generally shown at 10 of a transportable patient therapy and trolley assembly with a width adjustable carriage, see further at 12 which is laterally adjustable upon a width extending support or extrusion 14 incorporated into the trolley. The trolley 10 is further understood to include any upwardly extending body or structure (either fixed or mobile) which can support a robotic unit, as further generally depicted at 16, which is mounted in extending fashion below the width adjustable carriage 12 and terminates in a downward most probe head 18. Upon positioning a patient (not shown) underneath the trolley, with the probe head 18 adjusted to incrementally descend vertically into contact with the patient at a desired pressure variable, a separate processor control component (further shown in non-limiting representation at 20 with touch screen or other readout display 22) interfaces with the robotic unit, such as via numerical control (NC) inputs associated with the robotic unit, to provide the desired treatment protocol.

Without limitation, the processor 20 can include a single board computer (Raspberry pi), however can also include any of a plug in PC, laptop, or touch screen tablet/hand held smart phone incorporating a mobile application (such as relating to the software component protocols to be further described with reference to the screen depictions of FIGS. 1-2).

The trolley as depicted in the non-limiting variant includes a top width extending support 24, with first 26 and second 28 downwardly extending vertical supports in order to establish a generally “U” shape. As further shown, bottom pedestal supports 30 and 32 are integrated into the lower ends of the vertical supports 26/28 so that the bottom pedestal supports extend in both forward and rear directions to provide a stable base support for the assembly 10.

Without limitation, the repositionable trolley or carriage as shown can include without limitation such as an H frame structure which is designed to fit over any standard therapeutic table (not shown). Pairs of trolley wheels are depicted at 34 and 36 associated with each of the bottom pedestal supports to allow the patient care assembly to be moved into position so that the downwardly extending probe 18 of the robotic therapy unit is positioned above the patient (again not shown but understood to be supported upon any of a therapy bed or other support/positioning device). Any number of the trolley wheels can further include any type of manual lock or brake as is known for securing the assembly in an overhead position relative to the patient. A lock/unlock button 38 is positioned at an accessible location of the trolley body (see as shown by non-limiting example at an upper end of selected side extending support 28) and communicates with the trolley wheels (such as without limitation via solenoid actuated locks built into the wheels) in order to selectively engage or disengage the wheels. It is also envisioned that the lock/unlock button 38 can be substituted by other structure such as locking levers built directly into some or all of the trolley wheels to permit both ease of transport and anchored positioning of the trolley assembly and supported patient therapy robot.

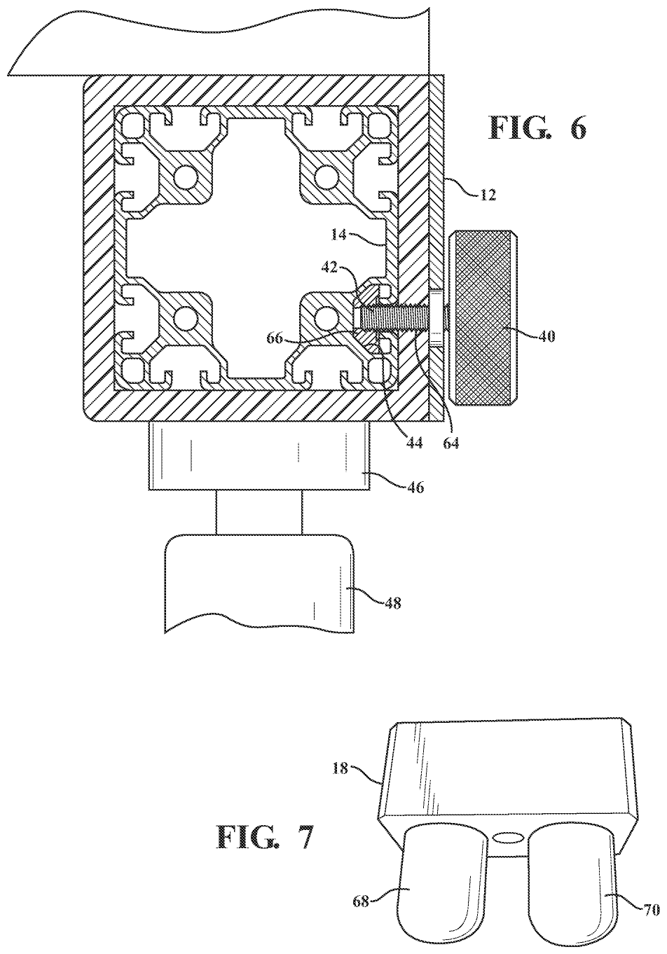

The width extending robotic unit therapy support 14 is also shown in the cutaway of FIG. 6 and can include any type of extrusion or other rigid support member which can provide both additional structural stability to the trolley assembly as well as reliably supporting and positioning the robotic therapy unit 16. As further shown in FIG. 6, a tightening knob 40 is provided with a threaded screw 42 extending through a face of the carriage 12, the screw 42 being received within a recessed and width extending channel (see at 44) in the support extrusion 14 and, upon positioning the carriage 12 at the desired width location between the horizontal side supports 26/28, the knob 40 is tightened to lock the carriage in place.

Additional components of the robotic therapy unit 12 include a fixed underside portion 46 which projects from the carriage 12 and which in turn supports the adjustment components associated with the robotic therapy unit. The robotic therapy unit 16 includes at least upper 48 and lower 50 inter-adjusting portions which, as will be further described, provide multi-positional adjustment of the underside extending probe head 18 relative to the patient being treated.

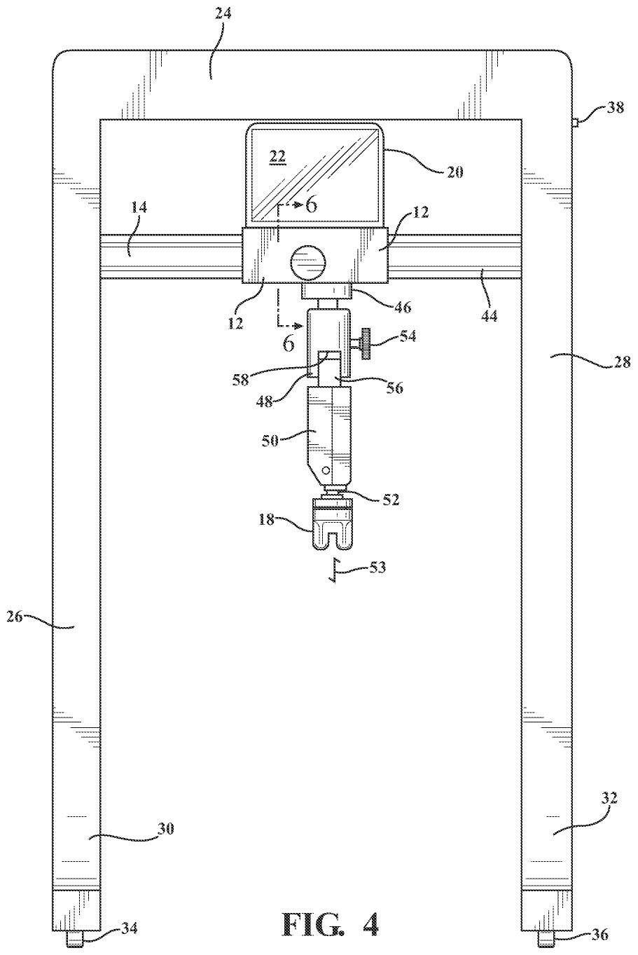

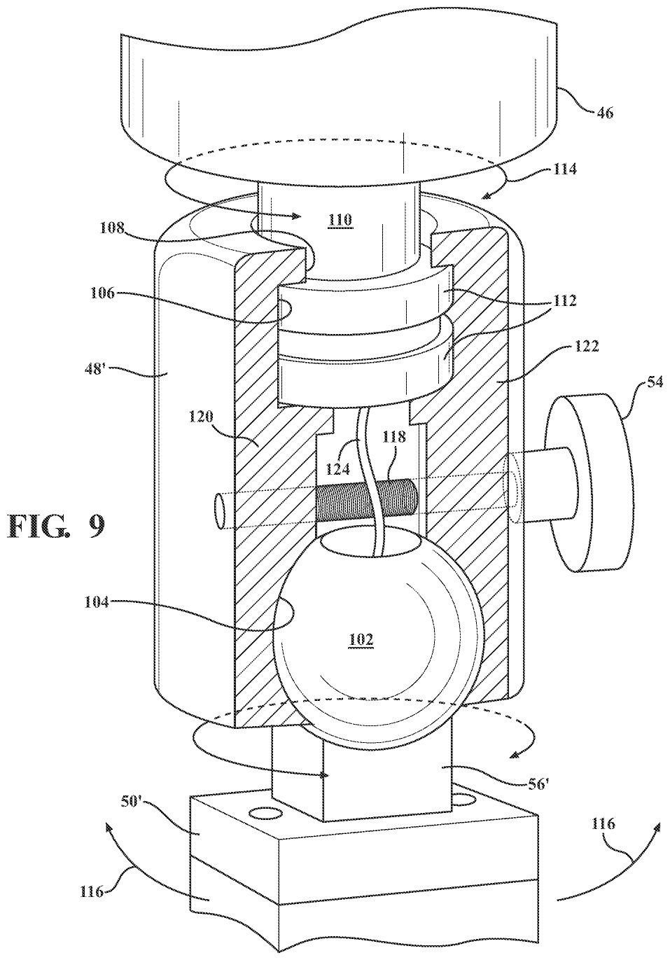

Without limitation, and as will be further described, this can include the upper portion 48 being swivel supported to the fixed underside portion, with the lower interconnected portion 50 being either pivotally or eccentrically adjusted (see also FIG. 9) in order to pre-position the probe head 18 relative to the patient being treated. Once positioned, the support structure for the probe head 18 (shown at 52) is further understood to incrementally adjust the probe head in an extensible direction (see arrow 53 in FIG. 4) to provide a tactile contact with the patient at the therapy treatment location.

As will be further described with reference to the related variant of FIG. 9, a further tightening/adjustment knob 54 is provided at a pivoting/eccentric interface established between the upper 48 and lower 50 interconnecting portions for adjusting the articulating relationship between the upper 48 and lower 50 portions. This can include (without limitation) an upwardly projecting neck 56 of the lower portion 50 being received within a central underside cavity 58 configured within the upper portion 48 such that the adjustment knob 54 permits the neck 56, lower portion 50 and probe head 18 to be pivoted relative to the upper portion 48.

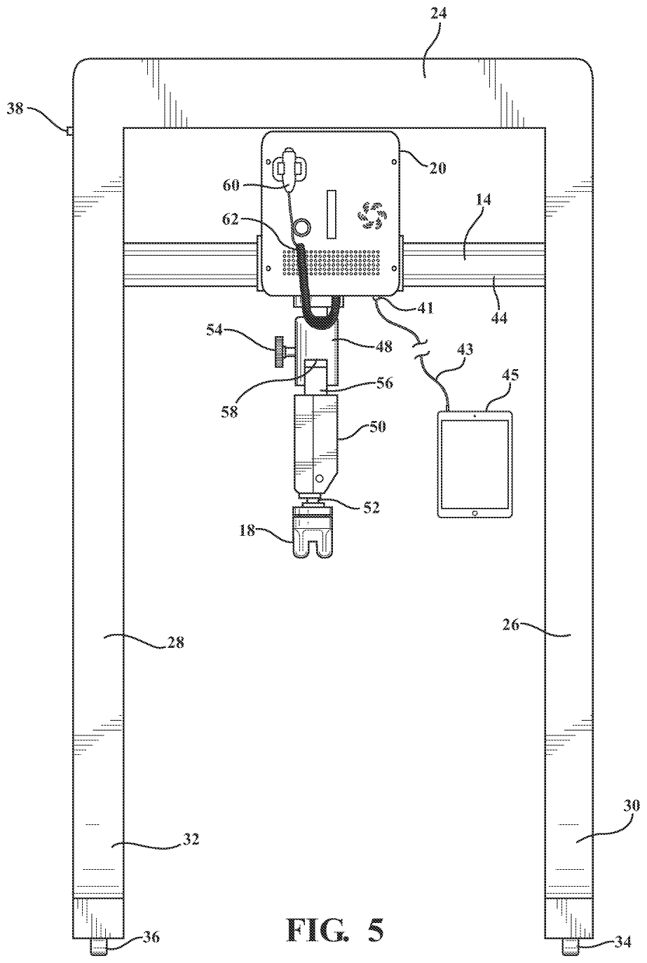

FIG. 4 is a front plan view of the trolley displaceable carriage and robotic unit of FIG. 3, with FIG. 5 further depicting a rear plan view of the trolley displaceable carriage and robotic unit and which also depicts the patient emergency stop button 60 for deactivating operation of the therapy probe head 18 (by stopping the application of the heat/cold, vibration, or other inputs and by optionally retracting the probe head the incremental distance 53 (FIG. 4) so that it is withdrawn from contact with the patient (not shown). Also shown in FIG. 5 is an extensible cord 62 which extends from the associated processor control (again generally represented at 20) and which can be held by the patient during therapy treatment.

FIG. 5 also depicts a direct location connection (such as a plug or inlet port 41) which is also configured somewhere along the robotic unit 16 and associated built in numerical controller 20). As further shown, any type of wireless connection protocol can be employed (again not limited to any of Wi-Fi, Bluetooth or NFC communication protocols) or, alternatively, a wired plug in connection connection (see as represented by cord 43) can communicate the inlet port 41 with a remote processor device, this further shown as a tablet style computer 45 however which is understood to include any type of laptop or other processing device. In this fashion, the incorporation of a direct local connection allows for localized transfer of data, such as to a processor device in the possession of the technician and which can include the ability to save or copy data associated with the patient treatment/therapy for any purpose.

FIG. 6 again provides a cutaway taken along line 4-4 of FIG. 4 and showing the tightening knob 40 for adjusting the lateral positioning of the carriage 12 in turn supporting the robotic unit 16 with adjustable probe 18. As shown in cutaway, the threaded shaft 42 associated with the knob 40 is received through an aperture location 64 of the outer carriage 12 (the carriage further depicted as a three sided and “U” shaped end profile article as referenced in FIG. 3). As further shown in FIG. 6, aligning and interiorly threaded locations are provided within a receiving nut having a pocket shape 66 which is supported within the recess channel 44 in the width extending extrusion 14 and which, upon tightening the knob 40, providing for securing of the carriage 12 at the desired width positioned location relative to the open underside of the “U” shaped portable trolley assembly.

FIG. 7 is an illustration of the patient therapeutic probe head 18 attachment to the extending end of the robotic unit 16 (FIG. 3). As shown, the probe heat attachment includes any number of therapy massage protrusions, see at 68 and 70, such that the probe head can delivery any combination of heat/cold, vibration, pulsing, etc., based upon the inputs to the robotic unit provided from the associate processor control (e.g. control panel). The functionality integrated into the probe head for providing the various temperature, vibration and other inputs is further understood to be consistent with the technology available for use in existing therapy massage/muscle lengthening assemblies known in the art such that a further technical explanation of its operation is unnecessary.

Consistent with the above, the probe head 18 can include internal thermocouple controlled resistor components, such as which assists in a correct delivery of heat at a specified temperature, and is further controlled for telescoping (downward) motion, such as which is provided by an appropriate servo drives integrated into the assembly and which are responsive to either of operator manual input or software generated commands. Reference is again made to FIG. 3 et seq. as to the arrangement of other structures associated with the support carriage and robotic assembly, such as which can be reconfigured in any manner desired according to the present inventions.

The probe 18 can be hollow and is designed specifically to house an oscillating motor and the electronic heat elements. The addition of the vibration components is intended to disrupt the nervous system's defensive response to tighten a muscle against pressure. The heat component is further added to aid in tissue relaxation and vasodilation and will aid in the detoxification of cell waste stored in the muscular tissue. Additional aspects include providing a lighter robotic unit and carriage by changing the materials used in manufacturing and reengineering the structure itself.

In this fashion, the probe is designed to apply static pressure to the musculoskeletal tissue in intervals, the static pressure again allowing for the non-surgical lengthening of muscular tissue, as well as the breakdown of adhesions or scar tissue that may be present. As further previously described, the overall purpose of the lengthening process is to allow a shortened muscle to return to its proper length, thus taking tension off of the bone structure and nerves.

Proceeding to FIG. 1, the non-transitory software component is shown to include a title screen illustration 72 of an associated software component incorporated into the robotic therapy device and which illustrates a user login screen with ID 74 and password 76 fields. FIG. 2 is a treatment set up screen 78 succeeding the login screen and which includes a plurality of touch entry screens for establishing parameters (identified as percentage for each of pressure 80, vibration 82 and heat 84), with an additional field for treatment duration 86 (in minutes). Additional indicated fields are provided for each of manual adjustment (up 88 and down 90) and such as for incremental extension of the probe head 18, along with start 92, stop 94, and logout 96 fields.

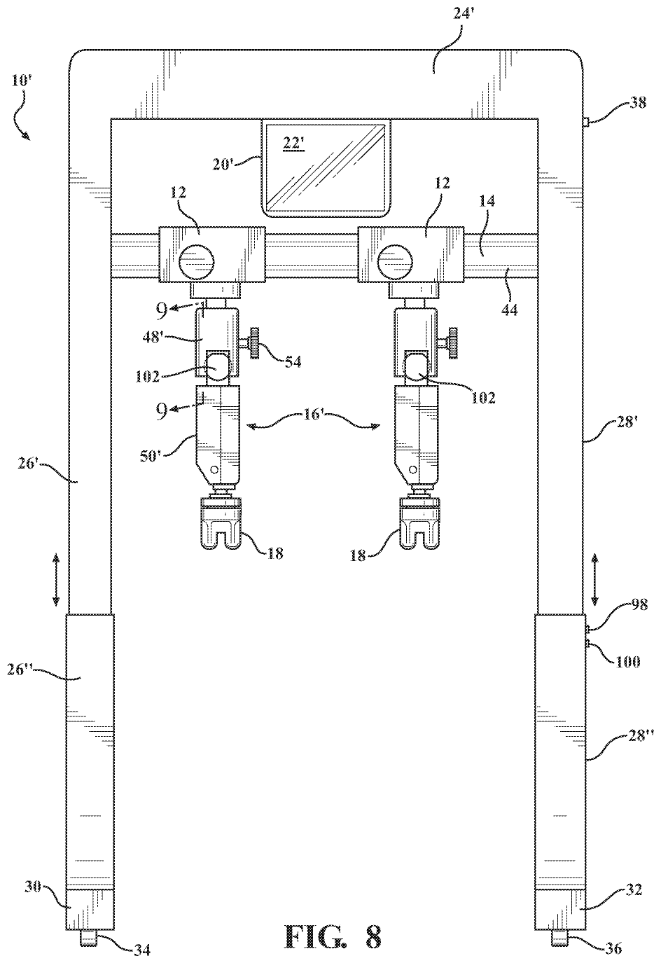

Having described a basic version of the present assembly, FIG. 8 provides a front plan view illustration of a trolley displaceable carriage and robotic unit according to a further variant presenting a pair of carriage supported robotic units 16′ with adjustable probes supported in underneath extending fashion from individual carriages 12 in turn supported upon the width extending extrusion 14. Also shown is a height adjustable trolley frame, generally further at 10′.

Identical components to the version 10 of FIG. 3 are repetitively numbered and description will be limited to the revised features which include the trolley frame being reconfigured with overhead cross support 24′ integrating reconfigured pairs of telescopic adjustable members (see sides 26′/26″ and 28′/28″). Adjustment buttons (up 98 and down 100) are indicated on telescoping support 28″ for incrementally adjusting the upper trolley and robotic units 16″ vertically upon pre-positioning the trolley over the patient (such as again supported upon the treatment table or other patient support device.

Also depicted are pairs of upper 48′ and lower 50′ interconnecting portions associated with the pair of robotic units 16′ (compare to as previously described in FIG. 3). The extending neck 56 associated with the variant of FIG. 3 is reconfigured as 56′ with an uppermost spherical ball 102 in each robotic assembly 16′, this seating within a likewise spherical pocket recess (better shown at 104 in FIG. 9).

FIG. 9 is a cutaway taken along line 9-9 of FIG. 8 and illustrates the eccentric adjustability of the probe head 18 and lower extending portion 50′ relative to the underside extending base support of each of the carriage adjustable robotic units 16′. The upper extending portion 48′ includes an upper annular opening defined by an expanded annular profile 106 within an open interior of the upper portion 48′ in combination with an undercut and uppers most narrowed rim 108.

An underside neck 110 extending from the width adjustable carriage 12 further includes one or more annular expanded supports 112 which seat within the annular expanded interior of the upper interconnecting portion 48′ to permit rotational adjustment of the upper portion (see arrow 114), in combination with any eccentric adjustability of the lower interconnecting portion 50′ (see further arrows 116). As configured, the tightening screw 54 with interior threaded shaft 118 extends through mating and aligning threaded interior locations of split halves 120/122 of the upper portion 48′ and, upon tightening, draw them together to lock in place the rotated 114 and eccentric adjusted 116 positions for the probe head 18. Also shown is a wire 124 which extends through the interior of each robotic unit 16′ to the undermost located probe head 18 for providing any of heat or power thereto.

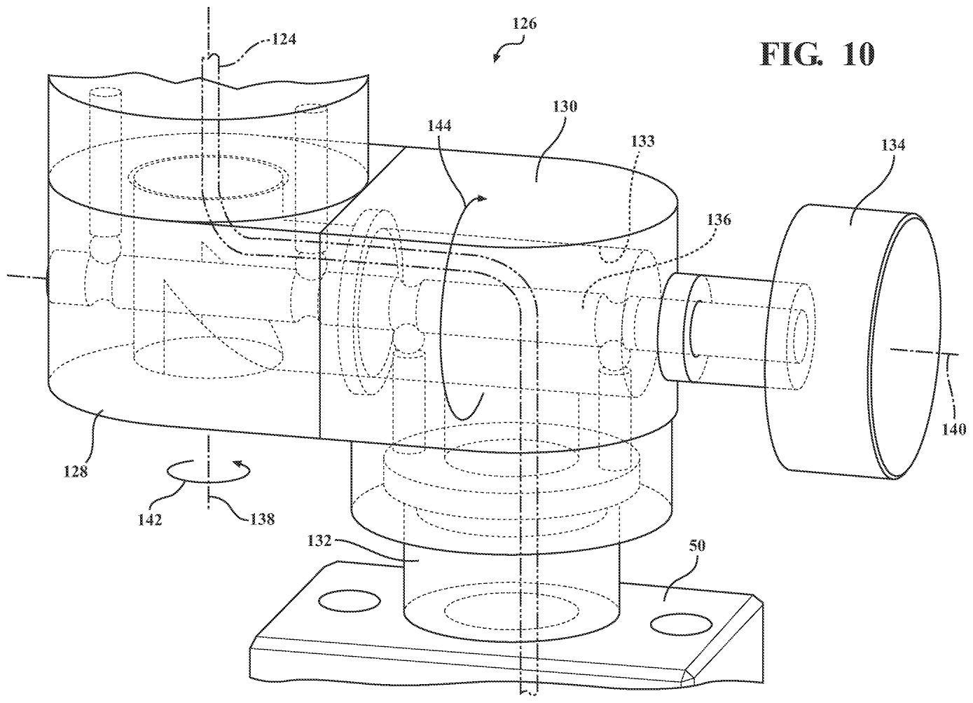

FIG. 10 is an illustration, at 126, of an alternate adjustment mechanism in comparison to that shown in FIG. 9 and depicting first 128 and second 130 rotational or swivel connected portions associated with a further reconfiguration of a carriage adjustable robotic unit for adjusting a positioning of the probe (not shown) relative to the patient being treated. The probe adjustment mechanism 126 is capable in one configuration to substitute for the upper 48 and lower 50 interconnected portions of FIG. 3.

Alternatively, the structure of FIG. 10 can be additionally provided at the underside support location (see again at 46) of the carriage 12 in order to provide additional axes of probe head adjustment, with the upper interconnecting portion (see again at 50) being indicated below an extending location 132 of the second swivel connected portion 130. A wire 124 extends from a remote power source and, in combination with the processor component 20, extends in a snaking fashion through a passageway (see inner wall 133 in phantom) extending within the interior of the robotic unit. In each of the embodiments disclosed, the wire is configured to avoid being kinked or damaged in response to the multi-axial adjustments of the probe head during execution of the programmed treatment protocol,

A tightening knob 134 is provided and, in combination with an elongated stem 136 supported within the interior, can be loosened and tightened (such as by an opposing thread arrangement as well as cam lock or other structures) in order to provide the configuration of FIG. 10 with the ability to further adjust the probe head about individual swivel axes indicated at 138 and 140 (and as further referenced by corresponding rotational directed arrows 142 and 144).

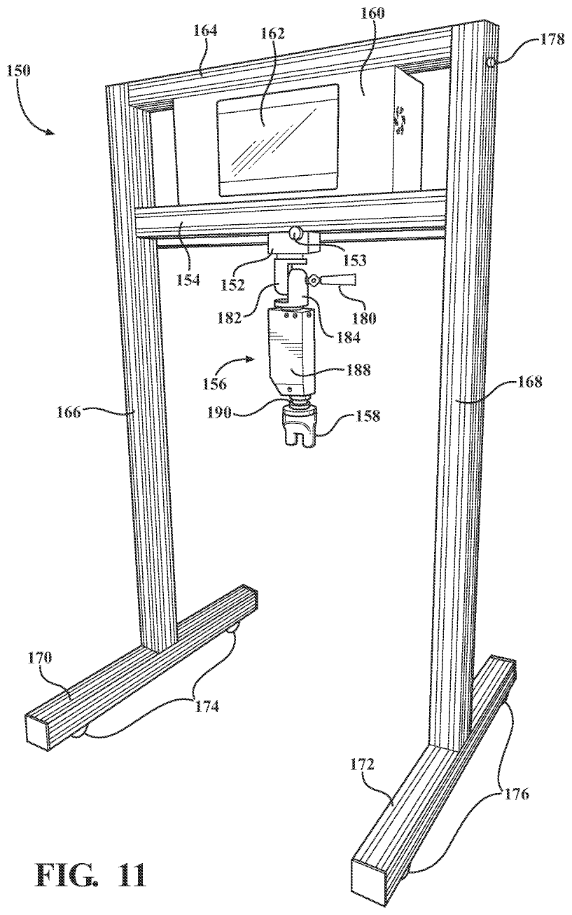

Referring now to FIG. 11, an illustration is generally shown at 150 of a portable transportable trolley with robotic therapy unit (similar in respects to that previously depicted in FIG. 3) and according to a further non-limiting and preferred embodiment of the present invention. As previously described in reference to FIG. 3, the transportable patient therapy and trolley assembly provides a width adjustable carriage, see further at 152, which is laterally adjustable upon a width extending support or extrusion 154 incorporated into the trolley (see also locking knob 153 for securing the carriage at a laterally adjusted position along width extending support or extrusion).

The trolley 150 is again further understood to include any upwardly extending body or structure (either fixed or mobile) which can support a robotic unit, as further generally depicted at 156, which is mounted in extending fashion below the width adjustable carriage 152 and terminates in a downward most probe head 158, such including any of a variety of different configurations however being illustrated to depict a pair of downward rounded projections or protrusions for applying a desired pressure to the patient. Upon positioning a patient (not shown) underneath the trolley, with the probe head 158 adjusted to incrementally descend vertically into contact with the patient (not shown) at a desired pressure variable, a separate processor control component (further shown in non-limiting representation as a cabinet or enclosure at 160 with touch screen or other readout display 162 positioned within a forward facing surface of the enclosure) interfaces with the robotic unit, such as again via numerical control (NC) inputs associated with the robotic unit, in order to provide the desired treatment protocol including the application of pressure, heat, vibratory effect and the like.

As previously described in reference to the initial variant of FIG. 3, the processor 160 can include any type of single board computer (such as without limitation a Raspberry pi type processor), as well as any of a plug in PC, laptop, or touch screen tablet/hand held smart phone incorporating a mobile application which can be wirelessly connected to the processor 160 (reference again being made to the software component protocols of FIGS. 1-2).

The trolley as depicted in the non-limiting variant includes a top or uppermost width extending support 164 a vertically spaced distance above the carriage supporting and width extending extrusion 154 for supporting the processor 160 and display 162 therebetween, with first 166 and second 168 downwardly extending vertical supports interconnecting the top width support 164 and lower width carriage support 154. Similar again to as previously shown in FIG. 3, bottom pedestal supports 170 and 172 are integrated into the lower ends of the vertical supports 166/168 so that the bottom pedestal supports extend in both forward and rear directions to provide a stable base support for the assembly 150.

Without limitation, the repositionable trolley or carriage as shown can again include without limitation such as an H frame structure which is designed to fit over any standard therapeutic table (not shown). Pairs of trolley wheels are depicted at 174 and 176 associated with each of the bottom pedestal supports allow the patient care assembly to be moved into position so that the downwardly extending probe 158 of the robotic therapy unit is positioned above the patient (again not shown but understood to be supported upon any of a therapy bed or other support/positioning device). A lock/unlock button 178 is positioned at an accessible location of the trolley body (see as shown by non-limiting example at an upper end of selected side extending support 168) and communicates with the trolley wheels (such as without limitation via solenoid actuated locks built into the wheels) in order to selectively engage or disengage the wheels. It is also envisioned that the lock/unlock button 178 can be substituted by other structure such as locking levers built directly into some or all of the trolley wheels to permit both ease of transport and anchored positioning of the trolley assembly and supported patient therapy robot.

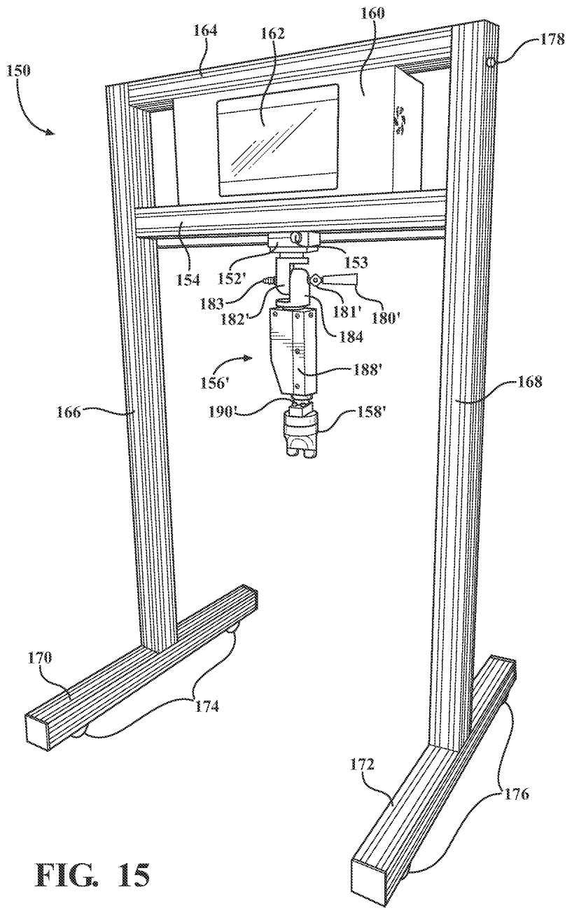

The width extending robotic unit therapy support 154 can again include any type of extrusion or other rigid support member which can provide both additional structural stability to the trolley assembly as well as reliably supporting and positioning the robotic therapy unit 156. Alternate to the tightening knob 40 in the preceding embodiment, a lock lever 180 is provided and which, upon being manipulated, includes interior structure such as a cam actuated stem (see as further described in subsequent variant of FIG. 15) to engage a first axial interface between first 182 and second 184 partially overlapping support portions, with the first support portion 182 being secured to an underside of the width adjustable carriage 152 and the partially overlapping second portion 184 in turn incorporating an end bearing 186 which rotatably supports a downward suspended main body 188 (see also at 50 in FIG. 3) of the robotic therapy unit, with the probe head 158 in turn secured in a multi-axial (eccentric) adjusted fashion to a lower projection 190 of the lower end of the main suspended body 188.

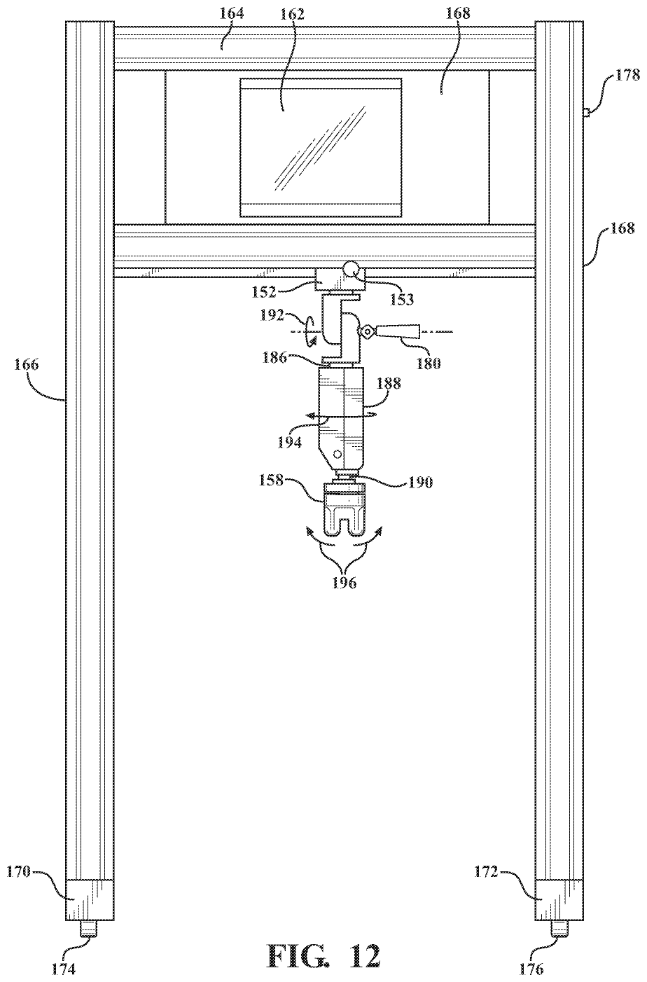

As with the variant 16 in FIG. 3, the robotic therapy unit 156 provides multiple axes of adjustment including at least as referenced at each of 192 and 194 in FIG. 12 and which correspond to a first tilting axis (192) established between the overlapping portions 182/184 and a second rotating axis (194) of the main body 188 and lower supported probe head 158. Without limitation, the probe head 158 can be fixed to the main body 188 of the robotic unit. Alternatively, the lower projection 190 can be reconfigured so as to define any type of eccentric adjustability, this further depicted by directional arrows 196. As previously described with reference to the first variant 16, the probe head 158 is typically pre-positioned relative to the patient being treated and, once positioned, the support structure for the probe head is incrementally adjusted in an extensible direction to provide a tactile contact with the patient at the therapy treatment location.

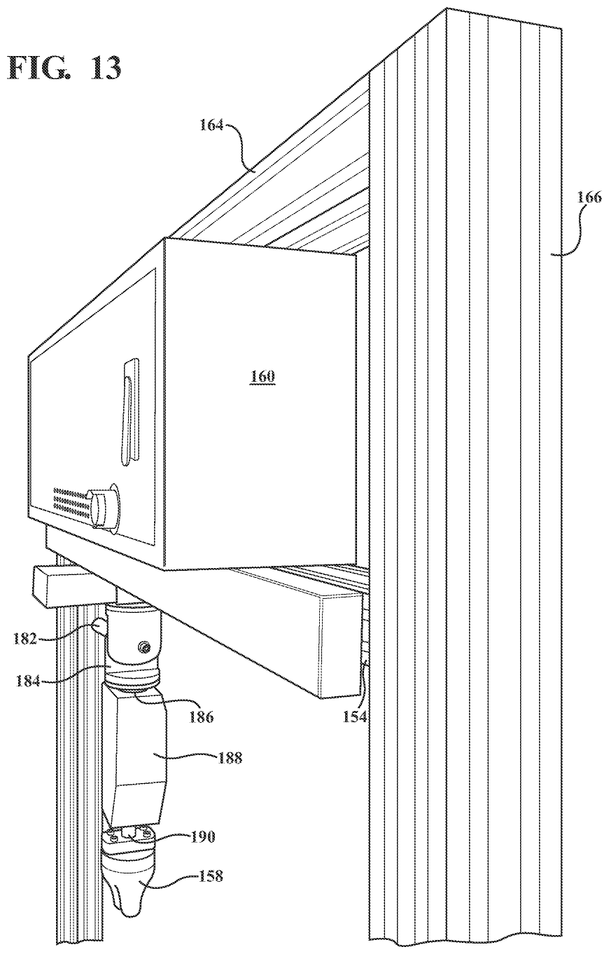

FIG. 12 is a front plan view of the trolley displaceable carriage and robotic unit of FIG. 11, with FIG. 13 further depicting an upper rear side perspective of the processor component mounted atop the width extending support from which the robotic unit is suspended.

As with the prior embodiment, the carriage and robotic unit can also include a patient emergency stop button (previously depicted at 60 in FIG. 5) for deactivating operation of the therapy probe head (by stopping the application of the heat/cold, vibration, or other inputs and by optionally retracting the probe head the incremental distance, see again at 53 FIG. 4, so that it is withdrawn from contact with the patient (not shown).

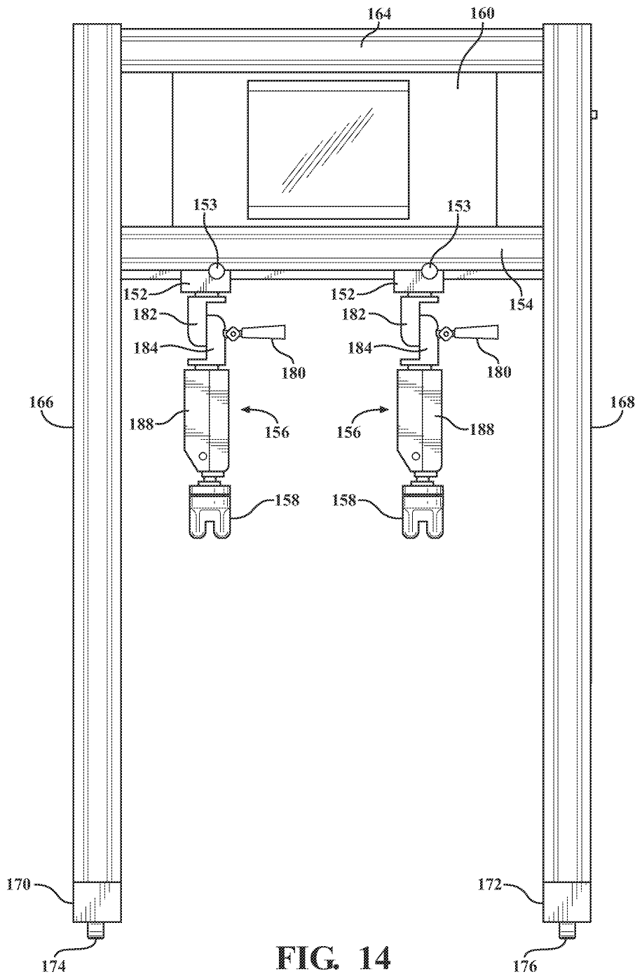

FIG. 14 is a front plan view similar to FIG. 12 and depicting a pair of carriage supported robotic units 156 as previously described, each with adjustable probe heads as previously described. Otherwise, the construction of the individual robotic units is substantially unchanged from that previously described.

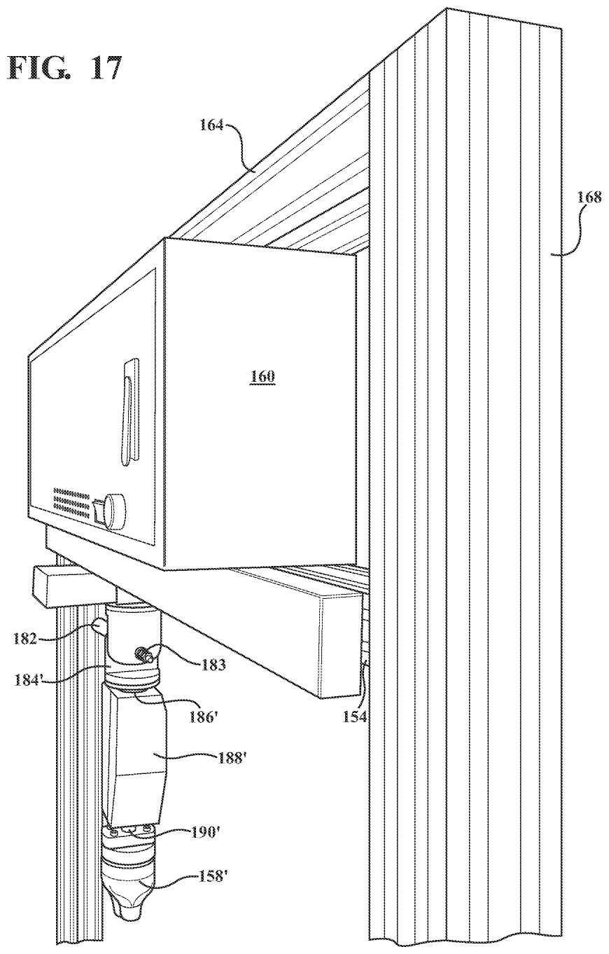

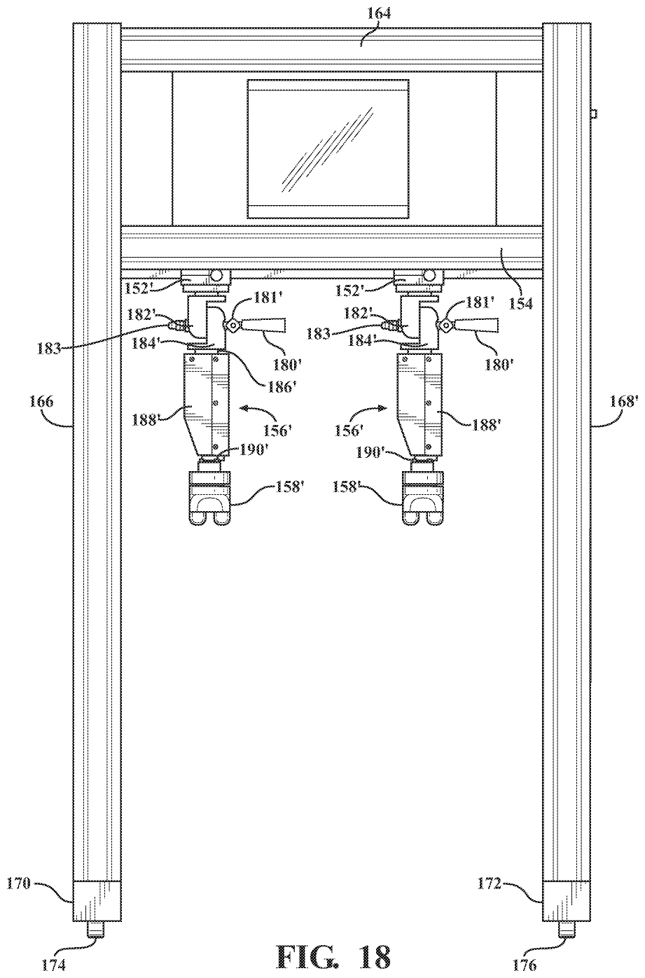

Referring now to FIGS. 15-18 substantially repeats FIGS. 11-14 while depicting some updated features of the portable therapy unit, in particular relating to the construction of the multi-axial robotic arm, shown at 156′ in single configuration in FIG. 15 and further in dual arrangement in FIG. 18 for supporting a downwardly extending probe head 158′. First 182′ and second 184′ partially overlapping support portions are again depicted which are locked in place via a pivotally associated handle 180′ which is pivotally actuated between an unlocked position and the rotated and locked position in which a cam actuation mechanism, see at 181′, is configured at a base of the handle and which, upon being manipulated, inter-actuates an internal stem or shaft (not shown) extending to a reverse end connected surface location (see nut end projection 183 associated with overlapping portion 182′) to compress together the overlapping support portions 182′/184′. An end bearing 186′ is located at an underside of the second overlapping portion 184′ and in turn rotatably supports a downward suspended main body 188′ (see also in comparison to that previously depicted at 188 in FIG. 11 and further at 50 in FIG. 3) of the robotic therapy unit, with the reconfigured probe head 158′ in turn secured in either of a fixed or axial (eccentric) adjusted fashion to a lower projection 190′ of the lower end of the main suspended body 188′.

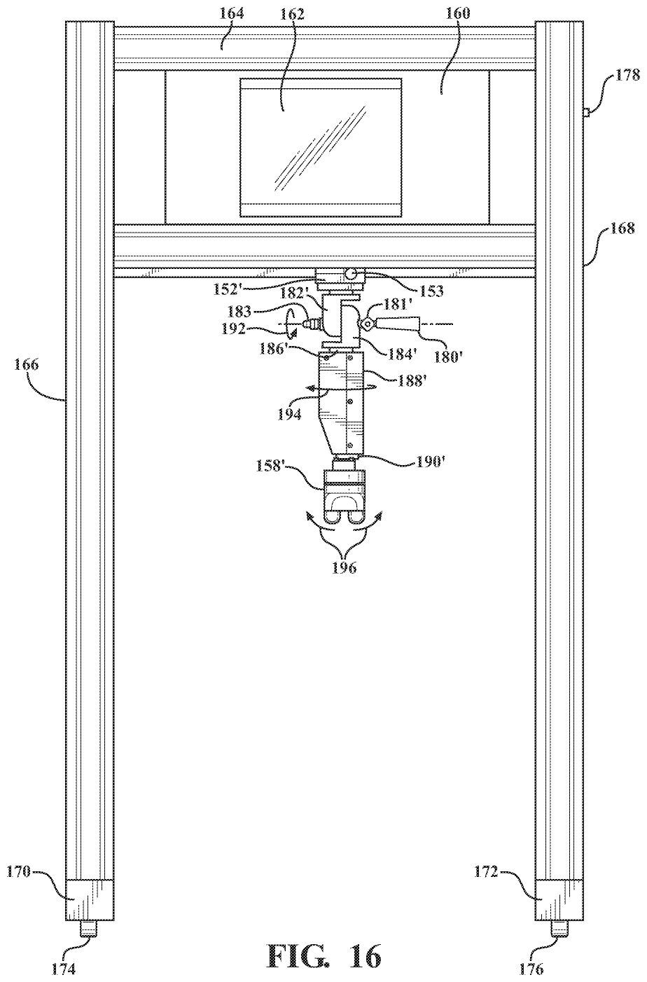

As with the variants 16 in FIGS. 3 and 156 in FIG. 11, the robotic therapy unit 156′ of FIGS. 15-18 provides multiple axes of adjustment including at least as referenced again at each of 192 and 194 in FIG. 16 and which correspond to a first tilting axis (192) established between the overlapping portions 182′/184′ and a second rotating axis (194) of the main body 188 and lower supported probe head 158. Without limitation, the probe head 158′ can be fixed to the main body 188 of the robotic unit. Alternatively, the lower projection 190 can be reconfigured so as to define any type of eccentric adjustability, this further depicted by directional arrows 196. As previously described with reference to the earlier variants, the probe head 158′ in FIGS. 15-18 is typically pre-positioned relative to the patient being treated and, once positioned, the support structure for the probe head is incrementally adjusted in an extensible direction to provide a tactile contact with the patient at the therapy treatment location.

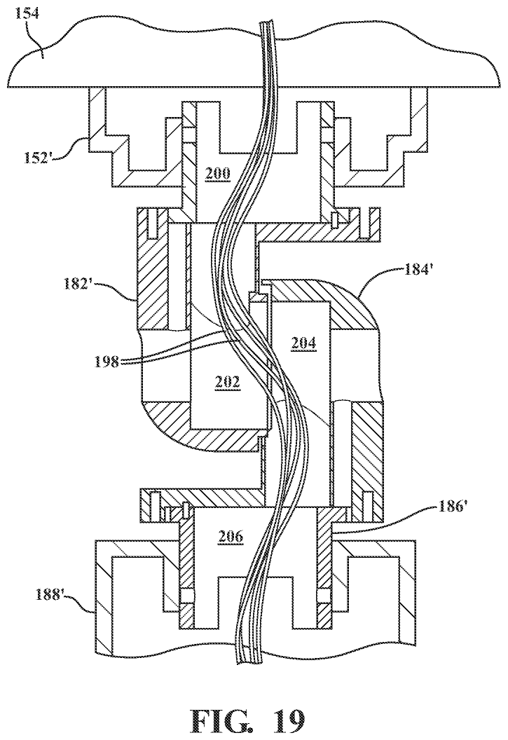

Proceeding now to FIG. 19, an enlarged plan cutaway of the robotic therapy unit providing multiple axes of adjustment and further illustrating a plurality of wires 198 (such as heater wires) extending from a heater component incorporated into an end location of the probe head 158′ (not shown in FIG. 19) to the processor 160 and display 162. The arrangement of the wires 198 is such they are not interfered with during the multi-axial adjustment (again at 192 and 194) of the individual articulating sections 182′/184′ and 186′ during manipulation of the probe head. Internal pathways are depicted extending through each of the widthwise supported carriage 152′ (at 200), within the pivotally interconnected overlapping portions 182′ (at 202) and 184′ (at 204) and, further, and the probe head end supported bearing 186′ (at 206).

Although not shown, it is envisioned that the wires 198 can terminate at the carriage 152′ or can extend into or through the widthwise support 14 of the frame to the processor control structure, such as where power to the heater wires can be provided (i.e. such optionally but not requiring a direct connection to the articulating robotic arms). Other options include additional contact wires integrated into the widthwise support 14 and the lower traversable carriage 152 for electrically communicating the upper mounted electrical box components of the processor control 160 to the lower probe head 158. This can also optionally include any of other type of direct wired or wireless configurations for providing any of heat, vibration, pressure etc., to the probe head. As also previously described, an interior compressing shaft is integrated into the pivoting handle lock 180′ and can extend through the overlapping internal pathways 202/204 as shown in FIG. 19 and in order to anchor to the opposite end surface mount 183 (again FIG. 15) and so that pivoting of the handle 180 or 180′ results in effective axial repositioning and locking of the lower support portion 184′ relative to the upper partially overlapping support portion 182′ and in order to array the lower probe head in the correct orientation.

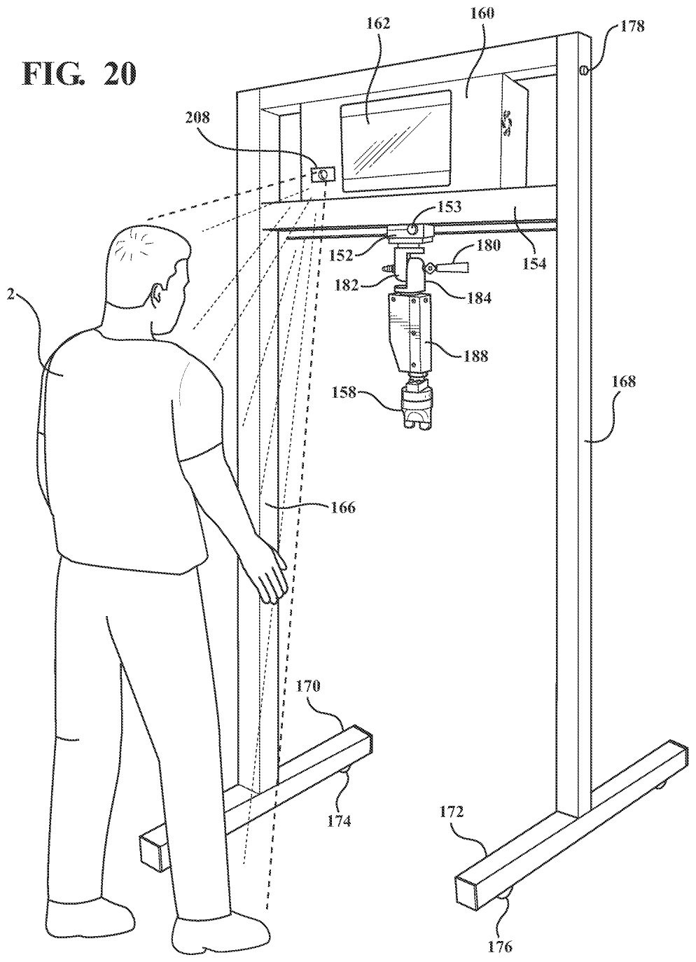

FIG. 20 is an illustration of a first version of a body scanner 208, which is depicted attached to a first location of the robotic therapy unit according to any of the previously described structural variants, and which interfaces with AI functionality in order to assemble a treatment protocol of a patient 2 standing in front of the unit. As understood, the AI algorithms are integrated within the software and hardware components of the robotic therapy unit as previously described in FIGS. 1-19, and which operate to provide each of body scanning for diagnostic and assessment points, establishing and implementing a treatment protocol for the patient which includes real time updated revisions in response to the AI algorithms, interfacing with the mobile/remote application for remote access of the data, interfacing with the software to auto-populate notations and records, and interfacing with the EMR's and other medical software components.

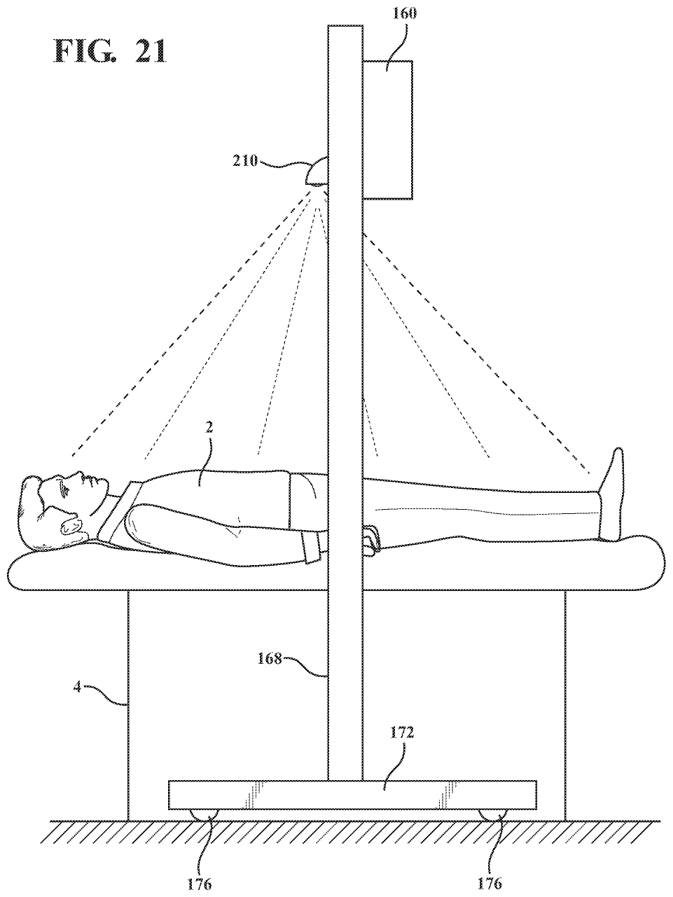

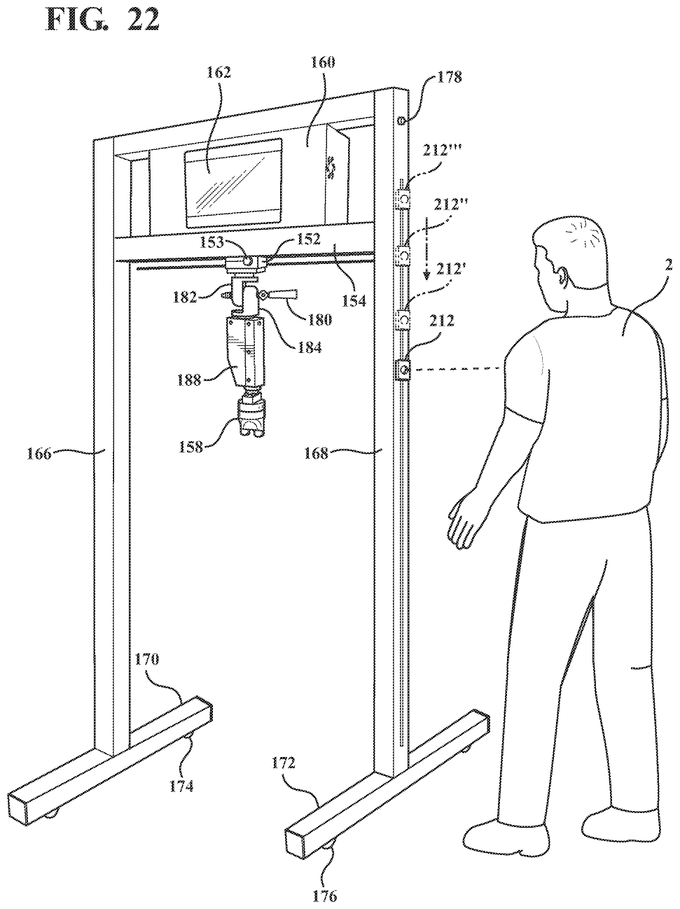

FIG. 21 provides a further illustration of a second version of a body scanner, at 210, in this instance attached to a downward facing location of the therapy unit scanner for conducting a scan of the patient 2 lying on a gurney 4 located underneath the associated carriage supporting the robotic therapy unit. FIG. 22 presents a third variant in which the scanner, at 212 is positioned at a side location of the robotic therapy unit carriage, such that it can be height adjustable to ascending locations 212′, 212″ and 212′″ depending upon the height of the patient 2 being scanned. Beyond that shown, it is also understood that the body scanner feature can be provided separately from the robotic therapy unit and support structure and this can include being integrated into a separate location not limited to being a portion of the associated mobile application integrated into a smartphone, tablet or the like.

As noted, additional features include the incorporation of a body scanner into the carriage assembly, this working in combination with the AI (artificial intelligence) features incorporated into the processor for conducting an initial body scan of the patient, at which point the processor assembles and outputs each of a detailed scan result and associated treatment protocol. As with the previously described embodiments, the operational aspects of the robotic therapy unit head 158 are assisted by the AI integration in the software to further provide for any one or more of ultrasound, frequency therapy, light/laser therapy, cold, heat, vibration, percussion, and mineral heads.

The AI features further can be incorporated into the mobile application as previously described for permitting remote access of the data, with software for assisting in auto-population of notations and records. This functionality also allows for generating the SOAP (subjective, objective, assessment and plan) notes as well as interfacing with electronic media records (EMR's) or other medical software.

The associated software based system disclosed herein is utilized in combination with the carriage and adjustable/numerically operable probe for providing the desired treatment protocol. As previously described, this can again include any of a single board processor (such as mounted or otherwise incorporated into the carriage) as well as any computer processor based input from any of a PC/desktop, laptop, tablet, or smart phone. As further understood, FIGS. 1-2 provide representative (as well as functionally and conceptually non-limiting) screen illustrations which can be incorporated into a keyboard and mouse operable PC, desktop or laptop computer, as well as incorporated into a capacitive touch screen associated with the tablet or smart phone. Without limitation, the present invention envisions other protocols which can be substituted or added to those shown.

As described, the above settings can be integrated into an automated program incorporating fixed settings for each of heat, vibration/frequency, duration, etc., and such as which can be selected according to approved best medical practice standards, such as formulated by the directed care provider, and to prevent the instances of medical malpractice or other misuse by the operator. Additional fields (such as which can represent customizable therapies which are authorized to the given operator) can be integrated into the associated software component and can include other fine adjustment options not limited to those depicted in FIG. 2.

The operating system disclosed allows the practitioner to set and change features such as probe pressure, rest intervals between pressure application, as well as varying intensity of vibration and heat via the probe head. Additional aspects of the design again include installed safety measures and fail-safes, such as again the patient or therapist operated handheld emergency stop 60 that will disengage the probe and lower the therapy table if activated.

The computer/software integrated aspects will also track which practitioner is operating the robot and which patient they are treating at that specific time. As referenced in the software module described herein, this activity is tracked via the entered individual ID and password assigned to each medical professional trained on our robot. This information is required to unlock the robot before use and will greatly reduce liability risk once paired with a patient PIN and any related notation software. It is further envisioned that all of the data stored in the robotic therapy unit will be able to be either uploaded to the cloud via wife, or downloaded via SD card, USB drive or cable, without limitation.

By way of example, one envisioned operating protocol can include treatment of a client who has a shortened pectoral muscle due to whiplash from a car accident. A physiological response to such a shortening would bring their shoulders forward causing the upper trapezius muscle in the back to shorten in attempts to balance the bone structure back out. The client would experience tension or pain the neck and shoulders, headaches, subluxations in the cervical and thoracic spine, bulging discs and many other symptoms.

By simply applying pressure via the robot to the pectoral muscle, it would allow the muscle to lengthen back out and the bone structure to realign. Once this process takes place the posterior muscles would no longer need to engage and very seldomly require additional treatment.

Another example would be a client suffering from a shortened hip flexor due to being seated for long periods of time. This condition is extremely common in the corporate world. A shortened hip flexor causes an anterior pelvic tilt as well as hyper lordosis in the Lumbar spine. Once the bone structure becomes imbalanced to this degree, the glutes, hamstrings and lateral rotators of the hip in the back tighten to offset the shortened hip flexor in the front. The client could experience low back pain, sciatica, bulging discs, hip pain, nerve impingement and many other symptoms. By simply applying pressure via the robot to the hip flexor, it would allow the muscle to lengthen back out and the bone structure to realign. Once this process takes place the posterior muscles would no longer need to engage and very seldomly require additional treatment.

Other envisioned variants include the ability to attach an add-on screen (such as a tablet via an additional bracket associated with the trolley) for use by the patient and which can provide some combination of inputs not limited to educational, marketing or entertainment features. The present system also provides the ability to complete SOAP notes (an acronym for subjective, objective, assessment, and plan) and which is a method of documentation employed by health care providers to write out notes in a patient's chart, along with other common formats, such as an admission note. In this manner, the associated process component (including without limitation such as a touch screen display) provides the user with the ability to extract data to populate medical notes or other data to any external software systems, network devices or the like.

Other variants can again include the use of any form of tablet, laptop or phone, such potentially being separately mounted to the support carriage and/or provided with a mobile application in communication with the software component, for providing any of NFC, Bluetooth or Cloud based directions to an associated numeric (NC) controller incorporated into the robotic system in order to program the probe to apply a treatment protocol according to the given combination of the heat, cold, vibration, pressure, pulse and time interval protocols. In this fashion, the probe head works in combination with a best practices protocol programmed into the associated software component, such as which is tailored to provide a desired treatment to a patient according to determined medical standards.

Other aspects of the present design again include the ability of the affixed or separate remote communicating processor to store and/or share secure patient records which may include but are not limited to treatment notes, and the like. Data sharing can further envision the use of any one or more of Cloud based, Messenger, USD/SD card, Mapping or 3D scanning options. The input/output aspects of the processor component associated with the probe can be communicated remotely via any of NFC (Near Field Communication), Bluetooth or Cloud capabilities with a remote care provider or ACO (or Accountable Care Organization which are defined as one or more groups of doctors, hospitals, or other health care providers, to provide coordinated care to a given patient group.

The practical outcomes of such treatments made possible by the present assembly include, without limitation, such as the dilation of blood vessels (vasodilation), such as in order to decrease blood pressure, realigning bone structure, breaking up adhesions, scar tissue and the like, and interrupting the physiological nervous response to defensively tighten/shorten the muscles.

Having described my invention, other and additional preferred embodiments will become apparent to those skilled in the art to which it pertains, and without deviating from the scope of the appended claims. The detailed description and drawings are further understood to be supportive of the disclosure, the scope of which being defined by the claims. While some of the best modes and other embodiments for carrying out the claimed teachings have been described in detail, various alternative designs and embodiments exist for practicing the disclosure defined in the appended claims.

I claim:

1. A combination software and robotic system for providing therapeutic treatment for a patient, comprising:
a processor control incorporating artificial intelligence assisted software algorithms communicating with a robotic unit for providing a series of instructions to a probe integrated into said robotic unit, a structure supporting said robotic unit which is adapted to being positioned in contact with a treatment location of a patient;
a body scanner incorporated into the processor for conducting an initial body scan of the patient, at which point the processor assembles and outputs each of a detailed scan result and associated treatment protocol; and
said instructions being provided, according to the scan results and treatment protocol, to said processor control in order to permit said robotic unit and probe to be controlled remotely for applying treatment to a patient muscle area according input parameters selected from at least one of heat, vibration, pressure and duration.

2. The combination software and robotic system of claim 1, further comprising said body scanner being configured within any of a front, side or downwardly facing location associated with said structure.

3. The combination software and robotic system of claim 1, further comprising a width extending support of said structure to which is secured an adjustable carriage supporting said robotic unit;

4. The combination software and robotic system of claim 1, said robotic unit further comprising a lock lever which engages a first axial interface between first and second overlapping support portions, said first support portion being secured to an underside of said adjustable carriage and said overlapping second portion in turn incorporating an end bearing which rotatably supports a downward suspended main body, with lower projection of said main suspended body supporting said probe head at a lower end of said robotic unit and so that said probe head is repositionable along separate axes of adjustment.

5. The combination software and robotic system of claim 4, further comprising a plurality of wires extending from said probe head through said robotic unit including interconnecting interior pathways defined in said overlapping support portions and said end bearing to communicate with said processor control for manipulating said probe; and

6. The combination software and robotic system of claim 1, further comprising a software component incorporating said artificial intelligence assisted software algorithms which interfaces with a touch screen display unit incorporated with said processor control into a cabinet mounted above said width extending support.

7. The combination software and robotic system of claim 6, said software component further comprising a login screen and a succeeding treatment set up screen.

8. The combination software and robotic system of claim 6, further comprising said software component having the ability to complete SOAP notes.

9. The combination software and robotic system of claim 7, said software component further comprising a data extraction protocol to populate medical notes taken by a care provider for communication to an external software system or network device.

10. The combination software and robotic system of claim 1, said processor control further communicating with said robotic unit by any of USB card, SD card, 3D scanning, text messaging application, short range wireless communication, Near Field Communication or loud based protocol.
